(19) United States
(12) Patent Application Publication
(10) Pub. No.: US 2006/0094777 A1
(43) Pub. Date: May 4, 2006

(54) Teat disinfectant having zinc EDTA, and methods

(76) Inventor: Geoffrey J. Westfall, Brooklyn, CT (US)

Correspondence Address: MERCHANT & GOULD PC, P.O. BOX 2903, MINNEAPOLIS, MN, 55402-0903, US

(21) Appl. No.: 10/978,503

(22) Filed: Nov. 1, 2004

(51) Int. Cl. A61K 31/315; A61K 31/155

(52) U.S. Cl. 514/494; 514/634

(57) ABSTRACT

Compositions, systems, and methods for the prevention of mastitis in milk producing animals are provided. In particular, the compositions comprise zinc EDTA and chlorhexidine. The composition is applied to the teat of a milk producing animal such as a cow either by dipping the teat therein or spraying the composition thereon. The composition is particularly suited for application by aerosol.

### FIELD OF THE INVENTION

The present invention is directed to the treatment or prevention of mastitis in milk producing animals. Specifically, the invention provides teat dip compositions and methods for application.

### BACKGROUND OF THE INVENTION

As a result of traditional selective breeding methods, milk production in dairy cows far exceeds the requirements of the newborn calf. Because of udder size, position, and anatomic configuration for rapid removal of large volumes of milk, the mammary glands of dairy cows are especially prone to injury and infection. In particular, mastitis, an infection of the mammary gland, is common in milking dairy cows, sheep, goats, and other milk producing animals.

Clinically, mastitis typically produces heat, swelling, tenderness and possible deformation of the udder. Although the milk from a mastitic udder is generally safe for human consumption, a major concern is the cost to producers. Mastitis causes a decrease in the amount and quality of milk produced by the infected cow. With decreased quality, the price obtained for the milk likewise decreases. Certain organisms, such as Escherichia coli, when associated with some mastitis, can lead to a cow's death.

Mastitis is typically caused by bacteria, such as Streptococcus agalactiae and Staphylococcus aureus, which enter the teat through the teat orifice. These bacteria account for about 90 percent of all udder infections.

Typically after milking, a drop of milk remains on the tip of the teat, which can serve as a nidus for bacterial growth. Also after milking, the teat canal is relaxed or dilated due to the vacuum applied by the milking machine. Bacteria can then migrate through the teat orifice and into the internal teat cistern to cause inflammation and blockages.

Mastitis causing bacteria can be spread among cows by contaminated milking units or machines, by cow to cow contact, or can be transferred by milking personnel having contaminated hands. General housing conditions, such as stall size, ventilation, bedding material, and access to pasture are also known to have an impact on mastitis spread.

Systemic and local antibiotic administration is a proven method of mastitis treatment and prevention. Intramammary administration of antibiotics during non-lactating periods, known as dry cow therapy, is an established method for mastitis prevention when a cow is not lactating. For cows that are lactating, teat dips have long been used as a preventative for mastitis. Post milking teat dipping is considered to be the single most important factor in mastitis prevention. Teat dips can function by providing a physical barrier to bacterial entry through the teat orifice. Additionally, bacteria that may be present can be killed by antibacterial ingredients of some teat dips.

Typically, the procedure of applying teat dip includes filling a cup or other suitable container with the dip formulation and dipping the teat therein. An aerosol spray generally may include the same or a slightly modified composition of the liquid dip and is sprayed on to the teat. The aerosol spray generally functions in the same manner as dips, with the exception that the aerosol can blow residual milk off the orifice such that there is no milk left to serve as a nidus for bacterial growth. The aerosol also has chilling effect on the teat, causing the sphincter muscle and teat orifice to contract, providing a further obstacle to prevent bacterial entry. Sometimes, a teat dip is applied with a pump sprayer.

Known active ingredients for teat dips include chlorine, iodine, in particular an iodophor, and chlorhexidine acetate and chlorhexidine gluconate. Some dip formulations have been developed which combine the desired germicidal or antibacterial properties of the active ingredients with suitable softeners or emollients, such as glycerin.

U.S. Pat. No. 6,183,785 to Westfall provides a teat dip that comprises a combination of chlorhexidine and zinc, the zinc being provided in salt form. Both the zinc and the chlorhexidine provide antibacterial properties. Although this and other well known and commercially available teat dips do have a beneficial effect on preventing the spread of mastitis, there is a continuing need for improved compositions and systems for treating and preventing mastitis.

### SUMMARY OF THE DISCLOSURE

The present invention relates to new and improved compositions, systems, and methods for the prevention of mastitis. In another aspect, the present invention relates to a new and improved composition for the reduction of the spread of mastitis.

In one embodiment, the present invention relates to new compositions for application to the teat orifice of a cow for prevention and treatment of mastitis. The composition includes zinc EDTA. Typically, zinc EDTA is present at a weight percent at least about 0.5% of the total composition. In a preferred embodiment, the zinc EDTA is present at a weight percentage of about 0.5%-5%, and preferably present at an amount of about 1%-1.5%. In another embodiment, the composition can comprise a combination of zinc EDTA and chlorhexidine. Typically, any chlorhexidine is present in the composition at a weight percent of about 0.1% to 4%. In a preferred embodiment, the chlorhexidine is present at a weight percent of about 0.5%.

In another embodiment, a system for preventing mastitis is presented. The system comprises a composition including a combination of chlorhexidine and zinc EDTA and a container for containing the composition therein. The container can be a cup, a jar or similar container, or can be a vessel from which the composition can be sprayed. For aerosol container, an aerosol propellant is preferably included in the composition.

In yet another embodiment, a containerized product for use in preventing mastitis is presented. A container capable of being pressurized has a disinfectant composition therein, the composition comprising chlorhexidine and zinc EDTA. Preferably, the composition is water based and comprises about 0.1% to 4% chlorhexidine, 0.5% to 5% zinc EDTA, and an aerosol propellant.

A method for preventing mastitis is disclosed, the method comprising preparing a composition having a combination of chlorhexidine and zinc EDTA, and applying the composition to the teat of a mammal, such as a cow. The composition can be applied to the teats of a cow by dipping the teats therein. Alternatively, the composition can be sprayed on to the teats, for example, as a liquid from a pump or as an aerosol.

### BRIEF DESCRIPTION OF THE DRAWINGS

FIG. 1 is a graphical representation of results from Test 1, in particular, the intramammary pathogen prevalence in the Control herd versus the Treated herd.

FIG. 2 is a graphical represented of results from Test 1, in particular, the means monthly milk production in the Control herd versus the Treated herd.

### DETAILED DESCRIPTION OF THE PREFERRED EMBODIMENT

Before the present composition and methods of use are disclosed and described, it is to be understood that this invention is not limited to the particular examples, compositions or methods disclosed herein, and that materials and methods may vary somewhat. It is also to be understood that the terminology used herein is used for the purpose of describing particular embodiments only and is not intended to be limiting.

The compositions and methods of the present invention are suitable for use with any milk producing mammals including, for example, cattle, sheep, goats, llamas, pigs, camels, etc. Because cattle are one of the most common milk-producing animals, the present invention is described with reference to dairy cattle. However, the invention should not be construed as being limited to cattle.

A single lactating dairy cow is capable of producing about 4 to 13 gallons of milk per day. Cows are typically milked at least twice per day to maintain health and milk production. During milking, milk from the udder flows into individual teats. In the teat, the milk flows into the teat cistern and then into the teat orifice, where it passes from the teat in a flow controlled by the teat sphincter muscle. Modern milking systems apply a pulsatile vacuum to the teat so that the sphincter muscle is intermittently opened and closed to release the milk.

To keep the cows healthy and to maintain good milk production, it is beneficial to take steps to prevent the onset of mastitis. One common preventative is application of a teat dip composition to the teats after milking.

The present invention provides teat dip compositions and methods for application to maintain hygiene and help prevent mammary infections. The compositions of the invention are typically applied to the skin of the teat, in particular to skin surrounding the teat orifice, as either a liquid (teat dip) or a spray, for example, an aerosol or by a pump. The composition of the present invention includes a zinc EDTA compound, and optional ingredients such as emollients, diluents, and dyes. In some compositions, zinc EDTA may provide adequate amounts of antibacterial protection, however, preferably, the zinc EDTA is combined with another antibiotic, antibacterial or other disinfectant component, such as chlorhexidine.

The present invention utilizes zinc ethylenediaminetetraacetic acid, commonly referred to as zinc EDTA. It has been found that zinc EDTA provides various benefits in the compositions of the present invention. It was discovered that zinc EDTA has antibacterial and/or antibacterial properties, which provide antibacterial and antimicrobial protection. Zinc EDTA also functions as a preservative, extending the shelf life of the composition, compared to compositions having other zinc salts.

It is well established that bacteria, such as Streptococcus agalactiae, Staphylococcus aureus, and Escherichia coli, are unable to grow and multiply without the presence of iron in the environment. Although not intending to be bound, it is believed that zinc EDTA, a known chelating agent, reacts with iron, if iron is present. In particular, the zinc ion on the EDTA is replaced with the iron ion. Thus, when a composition that includes zinc EDTA is applied to a surface (such as a teat), the EDTA scavenges for iron and removes the iron from the environment. Thus, the level of free iron in the environment decreases, due to the collection by the EDTA, and the level of free zinc increases, due to the release from the EDTA. This increased zinc and decreased iron concentration provides a ‘double-hit’ against infections, particularly mastitis. It is also believed that free copper would be chelated and bound by the EDTA.

The zinc EDTA compound is present in the composition at a level of about 0.01% to 10% by weight of the total teat dip composition, preferably 0.05% to 5%, more preferably about 0.25% to 4%, and most preferably about 0.5% to 1%. Zinc EDTA is a salt of EDTA, and is available as a solid or as a solution.

As stated above, zinc EDTA provides antibacterial and antimicrobial protection; however, preferably, the zinc EDTA is combined with another antibiotic, antibacterial or other disinfectant component, such as a chlorhexidine component.

If included, the chlorhexidine component of the composition is chlorhexidine or a salt thereof. Examples of suitable chlorhexidine salts include chlorhexidine gluconate, chlorhexidine acetate, and chlorhexidine diacetate. The chlorhexidine is typically present at a level of about 0.01 to 5% of the total teat dip composition, preferably 0.025 to 2.5%, more preferably about 0.1% to 2%, and most preferably about 0.25 to 1%.

The zinc EDTA and chlorhexidine, if present, are typically present in the composition at a ratio of about 1:2 to 2:1 zinc EDTA:chlorhexidine, preferably about 1:1. A composition being 0.5% zinc EDTA and 0.5% chlorhexidine is a preferred composition.

It has been found that is some formulations, zinc EDTA also provides antimicrobial or antibacterial properties. It is preferred to combine the zinc EDTA with a second antimicrobial compound, such as the chlorhexidine, although in certain formulations, zinc EDTA as the only antimicrobial or antibacterial ingredient may be sufficient in preventing or inhibiting mastitis. It is believed that EDTAs other than zinc may also have antimicrobial properties.

In addition to the antimicrobial or antibacterial ingredients, a lubricant or emollient can be added to the compositions, preferably in a range of about 0.5% to 20% of the composition. Suitable lubricants and emollients are well known and can be used for either dips or spray compositions. Water soluble emollients, such as glycerin (also referred to as glycerine), glycerol, sorbitol, and water-dispersible lanolin, are examples of suitable lubricants and emollients. Glycerin, at a level of about 10% by weight of the total composition, is a preferred additive.

Generally, the majority of the composition is an inactive or inert carrier, and is usually a liquid. The carrier may or may not act as a solvent for solubilizing the active ingredients of the composition (that is, the zinc EDTA and any chlorhexidine). Suitable liquid carriers include water (including tap water, distilled water, deionized water), and alcohol, preferably the lower alkanols of 3 carbon atoms or less, such as propanol, ethyl alcohol and methyl alcohol. The carrier is generally the largest portion of the formulation, typically present in a range of about 40% to 98% by weight, preferably about 70%. Alcohol, if present, is typically in a range up to about 70%; a preferred level for ethyl alcohol is about 10%.

The composition may also include additional optional additives, such as a pigment or dye, to act as an indicator whether a particular surface has been treated with the composition. Typically, a dye, for example FD&C Blue Dye #3, is included at a level of about 0.01% to 1.0%, preferably about 0.05% of the total composition.

The composition can be applied to the teat either as a “dip”, directly from a cup or similar container, or can be applied as a spray. If it is desired that the composition is applied as an aerosol spray, a propellant such as dimethyl ether, which can also function as a carrier, can be included. The propellant component, which is preferably water or alcohol soluble, may be present in a range of about 10% to 50%, preferably about 30%. An example of a preferred propellant is dimethyloxide, which is also referred to as dimethylether. It has been found that the combination of zinc EDTA and chlorhexidine is more stable when combined with a propellant, than is a composition of zinc and chlorhexidine.

Other components and additives may be optionally added, for example, stabilizers, and suitable corrosion inhibitors, which are particularly desirable when a tin plate container is used to dispense the composition or when a steel aerosol can is used.

The composition of the present invention may be prepared by mixing all the components, except the propellant, if propellant is used. If propellant is used, the mixture is poured in to a suitable container to be pressurized, after which the propellant is added under suitable pressurized conditions in accordance with conventional methods which are known. A container capable of being pressurized typically has a valve including an orifice nozzle discharge device capable of directing the disinfectant composition outwardly from the container.

In use, a composition of the invention will typically be applied to the teat immediately after milking, but may be additionally or alternately be used to sanitize the teat before milking.

The invention will be further described and illustrated in the examples that follow. The examples are illustrative of the invention and should not be construed as limiting the scope to their details. All parts, percentages, ratios, etc. are by weight unless otherwise specified.

### EXAMPLES

A composition was prepared having about 0.5 wt-% chlorhexidine, 0.5 wt-% zinc EDTA, 10 wt-% glycerin, 10 wt-% ethyl alcohol, about 0.05 wt-% FD&C Blue Dye #3, and the remainder distilled water. The composition was placed in aerosol spray cans with dimethyloxide aerosol propellant.

Test 1

A herd of dairy cows was collected (initially 85 total cows) and randomly divided into two herds: “Control” and “Treated”.

The Control herd had 43 cows, of which about 16.3% (7 cows) were initially infected with either coagulase-negative staphylococci (about 11.6%), coliforms (0%), Staphylococcus aureus (about 2.3%), or other Streptococci species (not including Streptoccocus agalactiae) (about 2.3%).

The Treated herd had 42 cows, of which about 21.4% (9 cows) were initially infected with either coagulase-negative staphylococci (about 14.3%), coliforms (about 7.1%), Staphylococcus aureus (about 4.8%), or other Streptococci species (not including Streptoccocus agalactiae) (0%).

The Control herd was treated with a 1% iodine composition, which was applied to the teats by dipping. The Treated herd was treated with the test composition described above.

Throughout the duration of the study (approximately 10 months), new cows were added and non-producing cows were removed.

At the end of the study, the Control herd had had 360 cows pass through, that, even after the 1% iodine treatment, about 15.6% were still infected with either coagulase-negative staphylococci (about 10.3%), coliforms (5.3%), Staphylococcus aureus (about 2.2%), or other Streptococci species (not including Streptoccocus agalactiae) (about 0.8%).

At the end of the study, the Treated herd had had 380 cows pass through, that, after the ZnEDTA/chlorohexidine treatment, only about 10.1% were still infected with either coagulase-negative staphylococci (about 6.3%), coliforms (5.5%), Staphylococcus aureus (about 0.5%), or other Streptococci species (not including Streptoccocus agalactiae) (about 0.5%).

These results show that the Treated herd had lower overall infection than the Control Herd, indicating that the combination of zinc EDTA with chlorohexidine was more effective at inhibiting and treating infection than iodine.

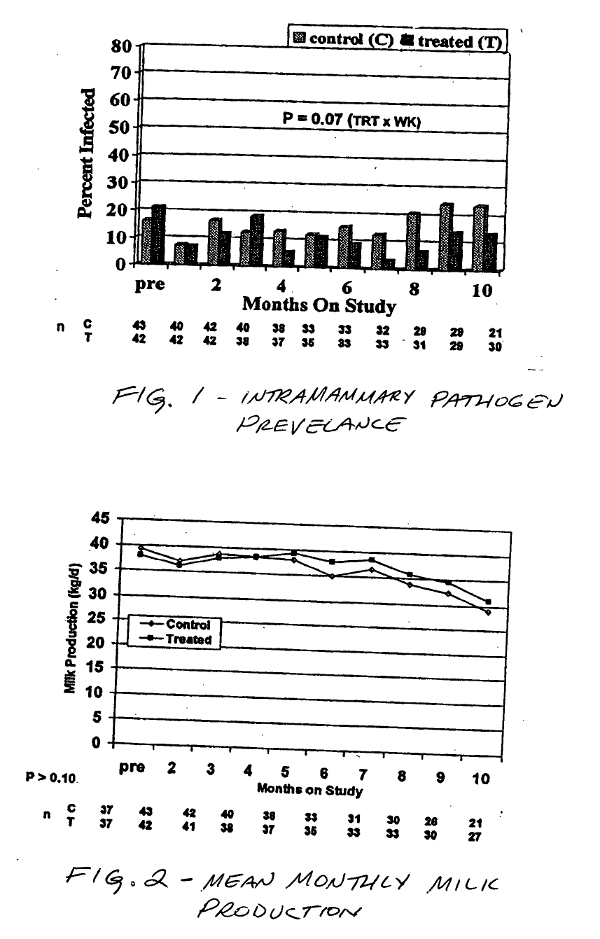

The results from the study, when plotted monthly over time, FIG. 1, showed that the Treated herd generally had a lower number of infected cows. The number of cows in each of the herds, monthly, is also shown in FIG. 1.

Test 2

A herd of dairy cows was collected (initially 73 total cows) and randomly divided into two herds: “Control” and “Treated”. The cows were rated according to the California Mastitis Test (CMT), which subjectively qualifies the level of mastitis infection as “0”, “trace”, “1”, “2”, of “3”.

The Control herd had 37 cows, which were initially rated as indicated in Table 1. The Treated herd had 36 cows, which were initially rated as indicated in Table 1.

The Control herd was treated with a 1% iodine composition, which was applied to the teats by dipping. The Treated herd was treated with the test composition described above.

Throughout the duration of the study (approximately 10 months), new cows were added and non-producing cows were removed.

At the end of the study, the Control herd had had 338 cows pass through, that, even after the 1% iodine treatment, were rated as indicated in Table 1. At the end of the study, the Treated herd had had 355 cows pass through, that after the ZnEDTA/chlorohexidine treatment, were rated as indicated in Table 1.

TABLE 1

| | |
|---|---|
| Initial | After Study Period |

| | Control | Treated | Control | Treated |
|---|---|---|---|---|
| CMT Score | (n = 37) | (n = 36) | (n = 338) | (n = 355) |
| 0 | 70.3% | 69.5% | 53.3% | 61.9% |
| trace | 13.5% | 11.1% | 21.0% | 17.3% |
| 1 | 5.4% | 11.1% | 9.2% | 7.7% |
| 2 | 8.1% | 5.6% | 8.9% | 8.5% |
| 3 | 2.7% | 2.8% | 7.7% | 4.6% |

These results show that the Treated herd had lower overall lower level of mastitis than the Control herd, indicating that the combination of zinc EDTA with chlorohexidine was more effective at inhibiting and treating mastitis than iodine.

Milk production is directly related to the presence of mastitis. The weight of the milk obtained from the test cows was plotted monthly over time, and is shown in FIG. 2. The result showed that the Treated herd had improved milk production over time. The number of cows in each of the herds, monthly, is also shown in FIG. 2.

From the foregoing detailed description and examples, it will be evident that modifications and variations can be made in the products and processes of the invention without departing from the spirit or scope of the invention. Therefore, it is intended that all modifications and verifications not departing from the spirit of the invention come within the scope of the claims and their equivalents.

I claim:

1. A method for treating a mammalian teat, the method comprising:
applying a hygiene maintaining composition to the teat of a mammal wherein the composition comprises chlorhexidine and zinc EDTA.

2. The method according to claim 1 wherein the step of applying the hygiene maintaining composition is by dipping the teat into the composition.

3. The method according to claim 1 wherein the step of applying the hygiene maintaining composition is by spraying the composition on the teat.

4. The method according to claim 3 wherein the hygiene maintaining composition is sprayed on the teat using an aerosol propellant.

5. The method according to claim 1 wherein the zinc EDTA is present at a level of about 0.05 to 5 wt-% of the hygiene maintaining composition.

6. The method according to claim 1 wherein the chlorhexidine is present at a level of about 0.05 to 5 wt-% of the total hygiene maintaining composition.

7. The method according to claim 1 wherein the zinc EDTA and chlorhexidine are present at a ratio of 1:1.

8. The method according to claim 1 wherein the zinc EDTA is present at a level of about 0.5 wt-% and the chlorhexidine is present at a level of about 0.5 wt-%.

9. The method according to claim 1 wherein the mammal is bovine.

10. A method of reducing bacteria counts associated with milk producing animals, the method comprising:
(a) selecting a composition comprising chlorhexidine and zinc EDTA; and
(b) topically applying the composition to a teat of an animal.

11. The method according to claim 10 wherein the animal is a cow.

12. The method according to claim 10 wherein the step of applying the composition comprises spraying the composition from a container.

13. The method according to claim 10 wherein the step of applying composition comprises dipping the teat into a container.

14. A composition comprising chlorhexidine and zinc EDTA.

15. The composition according to claim 14 wherein the zinc EDTA is present at a level of about 0.05 to 5 wt-% of the total composition and the chlorhexidine is present at a level of about 0.05 to 5 wt-% of the total composition.

16. The composition according to claim 15 wherein the zinc EDTA and chlorhexidine are present at a ratio of 1:1.

17. The composition according to claim 15 wherein the zinc EDTA is present at a level of about 0.5 wt-% and the chlorhexidine is present at a level of about 0.5 wt-%.

18. The composition according to claim 14 wherein the chlorhexidine is selected from the group consisting of chlorhexidine gluconate, chlorhexidine acetate and chlorhexidine diacetate.

19. The composition according to claim 18 wherein the chlorhexidine is chlorhexidine gluconate.

20. The composition according to claim 14 further comprising an emollient.
